(19) United States
(12) Patent Application Publication
(10) Pub. No.: US 2013/0122057 A1
(43) Pub. Date: May 16, 2013

(54) Organophosphorous, Multivalent Metal Compounds, and Bioactive Glass Material Macromolecular Network Compositions and Methods

(71) Applicants: Venkat R. Garigapati, Southborough, MA (US); Matthew E. Murphy, Whiterock Hill (IE); Cassandra L. Kimsey, Hoboken, NJ (US)

(72) Inventors: Venkat R. Garigapati, Southborough, MA (US); Matthew E. Murphy, Whiterock Hill (IE); Cassandra L. Kimsey, Hoboken, NJ (US)

(21) Appl. No.: 13/673,490

(22) Filed: Nov. 9, 2012

Related U.S. Application Data
(60) Provisional application No. 61/558,214, filed on Nov. 10, 2011.

(51) Int. Cl. A61L 27/18; A61L 24/04

(52) U.S. Cl. CPC A61L 27/18; A61L 24/046
USPC 424/400; 424/722; 424/602; 424/688; 424/678; 424/693; 514/114; 424/549; 424/93.7; 156/326; 427/140

(57) ABSTRACT

Cements containing certain small molecule amino acid phosphate compounds such as phosphoserine and certain multivalent metal compounds such as but not limited to calcium phosphate have been found to have improved properties and form a macromolecular network in the presence of a bioactive glass material that contain silicates, phosphates, and calcium salts which can be involved in the formation of bonding sites.

### CROSS REFERENCE TO RELATED APPLICATIONS

The present application claims benefit of U.S. Provisional Application No. 61/558,214 filed on Nov. 10, 2011, the entire contents of which are incorporated herein by reference.

### REFERENCE REGARDING FEDERALLY SPONSORED RESEARCH OR DEVELOPMENT

Not applicable

### SEQUENTIAL LISTING

Not applicable

### BACKGROUND OF THE DISCLOSURE

1. Field of the Disclosure

Improved calcium phosphate cements are well tolerated by the body. These improved cements include a macromolecular network resulting from the reaction between a bioactive glass material, multivalent metal compound, and a phosphoserine-like compound, in an aqueous environment.

2. Description of the Background of the Disclosure

Calcium phosphate composites are used as bone substitutes and bone grafts. These calcium phosphate composites tend to form complexes primarily between calcium-based salts through charge interactions. These composites are used as general bone void fillers and generally lack the adhesive strength sufficient to adhere or fix bones together, for example, fractured surfaces. These prior compositions have insufficient chemical interaction between the calcium phosphate composite and the bone surface or other surface materials and lack sufficient strength to be used to attach bone to bone or bone to other materials.

Certain marine species, such as tubeworms and sand castle worms, rely on secreted proteins containing a high amount of the amino acid phosphoserine for adhesion mechanisms (“The tube cement of Phragmatopoma californica: a solid foam,” Russell S. Stewart, James Co Weaver, Daniel E. Morse and J. Herbert Waite, Journal of Experimental Biology 207, 4727-4734, 2004). The specific mechanism of the phosphoserine involvement with the proteins is not understood; however, phosphoserine has been reported to be responsible for a specific interaction with calcium containing hydroxyapatite of bone as disclosed in U.S. Patent Application Publication No. 2005-0217538A1. In this publication, the authors mention calcium phosphate cements modified with phosphoserine (from 0.5% to 5% weight of the formulation) to aid as a compressive strength and surface area modifier in the bone cement material. In this range (from 0.5% to 5% weight of the formulation) the cement does not exhibit appreciable bone adhesion properties. In addition, certain bioactive fibers have been used as adjuncts to calcium cements, These fibers include bioactive glasses capable of fostering a calcium phosphate layer, which promotes bone bonding to the material.

### SUMMARY OF THE DISCLOSURE

One aspect of the present invention relates to a bone restorative composition comprising a reactive mixture of a small amino acid phosphate species, a multivalent metal compound, and a bioactive glass material that contains ionic functional groups.

A still further aspect of the present invention relates to kit for restoring bone comprising: A) a first container containing a composition comprising (i) a small amino acid phosphate species comprising a compound of the formula,

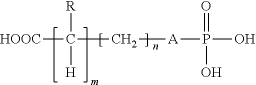

where A is O, CH₂, or S; R is H, NH₂, NHCO(CH₂)ₜCH₃ where t is 0 to 2, NH(CH₂)ₓCH₃ where x is 0 to 3, NR1R2 where R1 is (CH₂)_{y}CH₃ and R2 is (CH₂)_{y}CH₃ where y is 0 to 2, (CH₂)_{z}CH₃ where z is 0 to 3, where m is 0 to 1, and where n is 0 to 3; (ii) a multivalent metal compound; and (iii) a bioactive glass material that contains ionic functional groups, and B) a second container containing an aqueous medium.

Another aspect of the present invention relates to a method of adhering a substance to a bioactive glass material comprising the steps of: placing a composition between the material and the bioactive glass material wherein the composition comprises (1) small amino acid phosphate species comprising a compound of the formula:

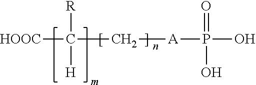

where A is O, CH₂, air S; R is H, NH₂, NHCO(CH₂)ₜCH₃ where t is 0 to 2, NH(CH₂)ₓCH₃ where x is 0 to 3, R1R2 where R1 is (CH₂)CH₃ and R2 is (CH₂)_{y}CH₃ where y is 0 to 2, (CH₂)_{z}CH₃ where z is 0 to 3, where m is 0 to I, and where n is 0 to 3; (ii) a multivalent metal compound, and (iii) an aqueous medium; and allowing the composition to cure to form an macromolecular network at the interface between the composition and the bioactive glass material, wherein the bioactive glass material contains ionic functional groups as the bonding sites of the bioactive glass material, and wherein the composition adheres to the substance.

A further aspect of the present invention relates to a method of repairing a hard surface comprising the steps of: mixing a composition comprising an effective amount of multivalent metal compound, a bioactive glass material, and a small amino acid phosphate species, wherein the small amino acid phosphate species comprising a compound of the formula:

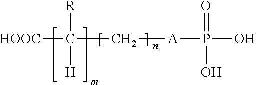

where A is O, CH₂, or S; R is H, NH₂, NHCO(CH₂)ₜCH₃ where t is 0 to 2, NH(CH₂)ₓCH₃ where x is 0 to 3, NR1R2 where R1 is (CH₂)_{y}CH₃ and R2 is (CH₂)_{y}CH₃ where y is 0 to 2, (CH₂)_{z}CH₃ where z is 0 to 3, where m is 0 to 1, and where n is 0 to 3, with an aqueous medium to create a mixture; applying the mixture to the hard surface to be repaired; and allowing the mixture to cure.

Other aspects and advantages of the present invention will become apparent upon consideration of the following detailed description.

### BRIEF DESCRIPTION OF THE DRAWINGS

FIG. 1 is a diagram showing detail of a proposed structure of the macromolecular network of the present disclosure.

### DETAILED DESCRIPTION

Small molecule multivalent metal compounds such as calcium phosphates, including tetracalcium phosphate (TTCP), react with small molecule organophosphate compounds such as amino acid phosphate compounds like phosphoserine to form cements, in the presence of aqueous environments, that have cohesive and adhesive properties. When these cements are in the presence of bioactive glass materials, the multivalent metal compounds and the amino acid phosphate compounds form a complex, macromolecular network with the surface of the bioactive glass materials. The significance of these macromolecular networks is the ability to form durable materials with high intrinsic strength (e.g. energy to failure) and in some cases extrinsic strength (e.g., adhesion to glassy surfaces). As used in this application, including the title and abstract, the term “bioactive glass” may be any alkali-containing ceramic, glass, glass-ceramic, or crystalline material that reacts as it comes in contact with physiologic fluids including, but not limited to, blood and serum. The bioactive glass material added to the formulation to form high intrinsic strength can be in any form, such as solution, powder, fiber, resin, liquid crystal, hydrogel, chip, flake, sheet, mesh, and the like. The glass based material to which the formulation can adhere can be in any form, such as plate, sheet, mesh, screw, pin, anchor, thread, fiber, suture, foam, film and the like. The complex formation of the macromolecular network system involves several modes of ionic or ion-dipole interactions stemming from the release of multivalent metal ions.

Certain small molecule amino acid phosphate compounds, such as phosphoserine, have a phosphate group (PO₄), a carboxyl group (COOH), and an amine group (NH₂) which are all capable of forming ionic interactions with the available metal ions. For rapid and abundant interactions, TTCP is the ideal metal ion source since it has the highest calcium to phosphate ratio (2:1) of the known calcium phosphate compounds and is well tolerated by the body. Basic TTCP is a calcium rich small molecule that is highly strained and dynamic. As it reacts in an acidic environment, the structure opens to release the calcium ions for ionic bonding. When it releases the calcium, the phosphoryl oxygen of the phosphate group of the TTCP intermediate is available for additional calcium ionic bonding. On this basis the authors hypothesize one method is to manufacture a calcium rich molecule with a calcium to phosphate higher then 2:1 which is even more reactive compared to TTCP. In addition, compositions with less reactivity can also be suitable for use. Such compositions could utilize calcium phosphate compounds with a calcium to phosphate ratio less than 2:1, such as alpha-tricalcium phosphate (1, 5:1) or compositions could utilize calcium based compounds which are not from the calcium-phosphate family, such as calcium chloride or calcium oxide. It is preferred that the multivalent metal compound be non-toxic as many uses of these compositions are for medical and/or veterinary uses. However, if the cement is not to be used relative to living organisms, toxicity is of less concern.

The bioactive glass comprises at least one alkali metal, such as sodium, potassium, and cesium, and combinations thereof. The bioactive glass may further comprise regions of combeite crystallite morphology. The bioactive glass used in the present disclosure reacts with the multivalent TTCP and the phosphoserine-like compound. Examples of such bioactive glasses suitable for use in the present disclosure are described in U.S. Pat. Nos. 5,914,356 and 5,681,872, each of which is incorporated by reference herein in its entirety.

One example of bioactive glass material is glass conventionally known as “45S5” glass in which combeite crystallites are formed. When combeite crystallites are fostered in the preparation of “45S5” bioactive glass such that combeite is present in amounts of at least about 2% by volume and preferably more, beneficial results are obtained. While not wishing to be bound by theory, this benefit may be in part due to the alteration of the stoichiometry of the base “45S5” glass, and subsequent reactivity and bioactivity, as the combeite crystal formation requisitions the selected ratios of ions. The residual amorphous content becomes increasingly higher in phosphorous (P₂O₅) content as the combeite crystal content increases. Improved bioactivity after crystallization is not a priori expected, as known bioactive glass-ceramics, such as A-W glass-ceramic, have reduced bioactivity upon increased crystallization from the parent glass.

It is highly desirable to foster the growth of combeite crystallites during the preparation of particulate, inorganic filler materials for use in biological restorations. It is desired to select starting minerals for preparation of the combeite glass-ceramics which contain the constituent elements of combeite and of the glass material which will form the amorphous regions surrounding the combeite crystallites.

It is desired to select starting materials for the preparation of the combeite glass-ceramics of the present disclosure which contain the constituent elements of the combeite, Na₄Ca₃Si₆O₁₆(OH)₂, and of the glass material Which will form amorphous regions surrounding the combeite crystallites. The glass material may be silicon, phosphate, or fluoride-based, or based on a mixture thereof, such as fluorophosphates. The overall constitution of certain preferred inorganic particles in accordance with the present disclosure, including both the glass portion and the crystallites of combeite, is as follows:

“45S5” glass has the following composition:
SiO₂ from 40% to 53% by weight, preferably from 43% to 48% by weight, and most preferably about 45% by weight;
Na₂O from 10% to 32% by weight, preferably from 15% to 30% by weight, and most preferably about 24.5% by weight;
P₂O₅ from 1% to 12% by weight, preferably from 2% to 10% by weight, and most preferably about 6% by weight; and
CaO from 10% to 32% by weight, preferably from 15% to 30% by weight, and most preferably about 24.5% by weight.
   This is the composition which is conventionally known as “45S5” glass. This “parent” or “base” glass has specific properties that are altered as a combeite crystal phase is sequestered.

Certain oxidation-reduction reactions take place during melting together with inorganic disproportionation such that the total constituency of the starting materials may be different from that of the product particles. In order to prepare “45S5” glass, it has conventionally known to employ 33.97% SiO₂, 30.98% Na₂O, 26.36% CaCO₃, and 8.68% CaHPO₄ (all percentages by weight). It will be appreciated that other formulations also give rise to the products the present invention and that all such formulations are within its spirit.

The preferred combeite glass-ceramic particles are such that at least about 95% by weight of said particles have particle sizes greater than about 0.1 microns. It is preferred that at least 95% of such particles have particle sizes greater than about 0.2 microns and less than about 710 microns. In some embodiments, the particle sizes are between about 10 to about 710 microns. In other embodiments, the particle sizes are between about 30 and about 90 microns. In yet other embodiments, the particle sizes are between about 90 and about 710 microns. Further, the distribution of the glass particle sizes may be bimodal. For instance, 50% of the combeite glass particles may comprise particles having a size between about 30 to about 90 microns, and 50% of the combeite glass particles may comprise from about 90 to about 710 microns. Other bimodal distributions may include from about 10% to about 90% of one range and from about 90% to about 10% of the other range, with a preferred distribution of about 50/50 of the two ranges.

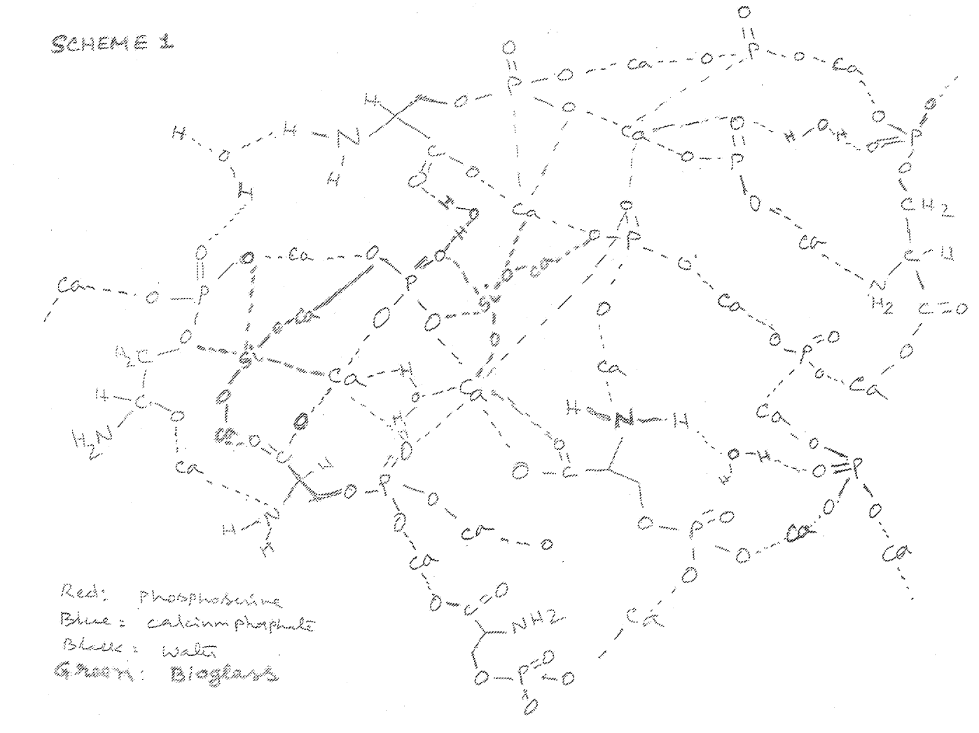

To create bioactive glass reinforced bone cement, ion-dipole multivalent metal ion interactions develop and are extended to the bioactive glass material. The electronegative ions and electropositive atoms are the bonding sites of the bioactive glass material. The reactive and charged TTCP/phosphoserine-like complex that develops influences the folding of the adjacent bioactive glass material in a favorable geometry to enhance the ion-dipole and hydrogen bonding. For example, the amine group and the oxygen atoms of the phosphoserine-like compounds bond with the charged species of the TTCP or similar compound and the bioactive glass material. Fourier Transform Infrared Spectroscopy (FTIR) data indicates that the functional groups of the phosphoserine-like compound and TTCP react rapidly when mixed with water. The reactive groups of the mixture further extend the reaction with the silicon and calcium ions of the bioactive glass to form a macromolecular network. This geometry is depicted in FIG. 1. The structure of FIG. 1 has been confirmed based on FTIR analysis.

FTIR data suggest that the —NH₂, —COOH and —P(O)(OH)₂ groups of the phosphoserine-like compound are involved in a reaction with the calcium ions of TTCP to form a hybrid organic/inorganic macromolecular network through calcium ion bridges. The incorporation of bioactive glass material interacts with the reactive species which are generated from TTCP and phosphoserine and result in the formation of a macromolecular network. The functional ions of the bioactive glass material react with both the TTCP and the phosphoserine-like compound to form the macromolecular network shown in FIG. 1. The functional ions of the bioactive glass material involve in reaction with reactive TTCP/phosphoserine-like compound to form calcium and silicon bridges and hydrogen bonding. Thus the bioactive glass material augments the strength of the TTCP and phosphoserine-like system by participating in the formation of an interpenetrating macromolecular network which is quite distinctive from the fiber reinforced calcium-based bone cements which are currently on the market. As the cure time of the aqueous solution progressed over 14 minutes, there were strong shifts in the FTIR spectrum showing a reaction among the TTCP, phosphoserine-like compound, and the bioactive glass material consistent with the structure shown in FIG. 1. Based on the FTIR data, the TTCP and phosphoserine-like compound react rapidly in water to form a macromolecular network with the bioactive glass material through calcium and silicon bridges and hydrogen bonding. Calcium phosphate cements without sufficient organophosphates such as phosphoserine do not have as much ability to influence this bioactive glass material folding effect; thus the ion-dipole interaction is not as strong and gives inferior intrinsic or extrinsic strength. One calcium ion in the macromolecular network system has the ability to interact with more than one electronegative ion from the bioactive glass material surface. An example of this ion-dipole interaction is with the calcium and silicon ions to form a coordinate complex [Rosetta Natoli Reusch and Harold L. Sadoff, Putative structure and functions of a poly-β-hydroxybutyrate/calcium polyphosphate channel in bacterial plasma membranes, Proc. Nat. Acad. Sci. USA, Vol, 85 [Jun. 1988] p. 4176-4180].

A bone restorative compound may alternatively comprise phosphoserine or similar compounds, bioactive glass material, water, and a calcium salt such as Ca(OH)₂, CaO, or CaCl₂. The phosphoserine reacts with the calcium and silicon ions of the bioactive glass material to form the macromolecular network even in the absence of TTCP. FTIR data suggest that the bioactive glass reacts with the —COOH functional groups of the phosphoserine-like compound to form a sticky mass. The sticky mass can be converted to a strong solid mass by supplementing divalent, trivalent, and polyvalent ions such as calcium, magnesium, strontium, barium, iron, aluminum, zinc, gold, titanium, platinum, or the like.

The macromolecular system of the present disclosure is similar to the interpenetrating network formed by TTCP, phosphoserine, and PLEA disclosed in U.S. application Ser. No. 13/104,716. One improvement of the composition of the present disclosure is that the use of bioactive glass materials does not involve any acidic functional groups. As a result, the bone cement of the present disclosure is more stable in a wider variety of environments.

It has further been found that certain multivalent metal compound cements that include a certain minimum amount of small amino acid phosphate compounds of the formula:

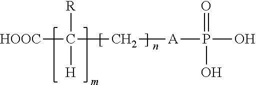

where A is O, CH₂, or S; R is H, NH₂, NHCO(CH₂)₁CH₃ where t is 0 to 2, NH(CH₂)ₓCH₃ where x is 0 to 3, NR1R2 where R1 is (CH₂)_{y}CH₃ and R2 is (CH₂)—)_{y}CH₃ where y is 0 to 2, (CH₂)_{z}CH₃ where z is 0 to 3, where m is 0 to I, and where n is 0 to 3, have superior strength in the presence of bioactive glass material that have ionically available electron pairs compared to known calcium phosphate or multivalent metal compound cements. Preferred adjunct compounds are those where A is O or CH₂, R is H or NH₂, m is 0 or 1 and n is 0 or 1. It is also possible that other similar materials that can assist the multivalent metal ionically bond to reactive sites on the resorbable bioactive glass materials are usable in the calcium phosphate mixture. At present one preferred species is phosphoserine.

The compositions as described herein are useful in a wide variety of medical applications. One use of the compositions is to adhere bone fragments together within the body. This is useful, for example, during surgery to allow for temporary fixation prior to definitive hardware placement, and to enhance fracture fixation by adhering both load and non-load bone fragments together alone and/or in the presence of appropriate immobilization. The compositions also enhance screw and/or bone anchor fixation into low density cancellous bone at and/or after surgery, to allow screw fixation when the core diameter of the screw hole is larger then the screw major diameter, for instance to reattach metal or bioresorbable screws to bone that has stripped from the surrounding material, to adhere a metal or bioresorbable plate to fractured bones allowing for reduction and/or elimination of metal or bioresorbable screws or pins used to fix a metal or bioresorbable plate to bone. The use of the compositions with a bioresorbable suture may be used to help fixate small bones after fracture. The compositions also have the capacity to enhance fixation of a joint replacement prosthesis to bone (e.g. hip acetabular cup or femoral stem). The compositions adhere the junction of at least one of a tendon, ligament, cartilage, a bone graft, and/or dental implants to bone. The compositions may be used to support new bone growth for dental socket or dental ridge augmentation. The compositions have the capacity to adhere to bony defect perimeters while filling gaps creating a seal to prevent leakage (e.g. cerebral spinal fluid). Furthermore, the compositions may also be used in ossicular chain reconstruction to adhere middle ear ossicles together. The adhesive properties of the compositions of the present disclosure to bone and bone to other materials make them useful to provide bony contour for facial bone augmentation applications. These compositions are also useful for gluing cancellous bones, cortical bones and a combination of both, whether in fatty or greasy environments potentially without any surface pretreatment prior to application.

One particularly novel use of the compositions is as a bone restorative composition. By a bone restorative composition, it is meant a composition that is useful to restore and/or repair bone, such as bone adhesives, bone cements, bone glues, bone putties, bone void fillers, bone replacement compositions, cements and/or adhesives to fix screws, metal or bioresorbable implants and at least one of a tendon, ligament, cartilage, a bone graft, and/or a dental implants to bone.

The compositions have a tacky state shortly after initial mixing with an aqueous medium. This tacky state enables at least two items, such as two pieces of bone, bone and another material, or two non-bone materials to be held together by the composition itself, without the need for external force, until the composition sets to the final hardened cement state. The amount of force needed to remove two opposed pieces of material from each other is the separation strength. For the composition as described herein, these compositions have a separation strength during the tacky state within the first 4 minutes and preferably within the first 2 minutes after initial mixing from about 10 kPa to about 250 kPa and preferably from about 50 kPa to about 150 kPa. For certain applications it may be useful to have a longer tack state whereby certain compositions have a separation strength which continues in this range for up to 12 minutes. This separation strength is sufficiently high that the items to be joined need not be held together unless there is an apposing strength of the items greater than the separation strength and also, the items can still be repositioned or even reapposed without loss of ultimate bond strength.

It has been found that in the present compositions TTCP has unusual properties not shared by other calcium phosphate compositions or other multivalent metal compounds. TTCP is the most basic of all the calcium phosphates; therefore, it readily reacts to acidic compounds. While other calcium phosphate compositions can be used in addition to the TTCP, it is preferred that the compositions include an effective amount of TTCP. The TTCP used in the present compositions can be made by a variety of methods. One such manufacturing method is disclosed by Chow and Takagi in U.S. Pat. No. 6,325,992, the disclosure of which is hereby incorporated by reference. The TTCP can be 100% pure material or can include other calcium and calcium phosphate materials as an impurity, e.g., α-TCP, CaO and/or HA.

Typical amounts of multivalent metal compounds are from about 5% to about 90% by weight based on the total composition of the dry ingredients, namely the metal compound, the small amino acid phosphate species and the bioactive glass material. A preferred and optimum amount of the metal compound is from about 40% to about 80% and about 40% to about 65% by weight on the same basis. Multivalent metal compounds may include calcium phosphate, magnesium phosphate, barium phosphates, strontium phosphate, titanium phosphate, zirconium phosphate, calcium oxide, magnesium oxide, and mixtures thereof. In addition, non-phosphate compounds of these metals such as carbonates, bicarbonates, sulfates and nitrates can replace some or all of the above phosphate compounds. Further examples of non-phosphate compounds include calcium oxide, calcium hydroxide, and calcium chloride. In addition, suitable multivalent metal compounds include a combination of cations and anions, with examples of suitable cations being: calcium, magnesium, barium, strontium, iron, zinc, titanium, zirconium and mixtures thereof, and anions being; phosphates, oxides, carbonates, bicarbonates, sulfates, hydroxides, chlorides, acetates, fatty acid salts, acetylacetones, and nitrates and mixtures thereof. In one embodiment, to obtain a reactive material having cohesive properties, such multivalent metal compounds include cations such as calcium, strontium and magnesium with anions such phosphates, oxides, hydroxides and chlorides. Multivalent metal compounds are discrete compounds that are not present as bioactive glass material.

A second component of the compositions is a small amino acid phosphate species that has the following formula:

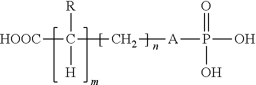

where A is O, CH₂, or S; R is H, NH₂, NHCO(CH₂)ₜCH₃ where t is 0 to 2, NH(CH₂)ₓCH₃ where x is 0 to 3, NR1R2 where R1 is (CH₂)_{y}CH₃ and R2 is (CH₂)_{y}CH₃ where y is 0 to 2, (CH₂)₂CH₃ where z is 0 to 3, where m is 0 to 1, and where n is 0 to 3. Preferred compounds are those where A is O or CH₂, R is H or NH₂, m is 0 or 1 and n is 0 or 1.

The most preferred small amino acid phosphate species is phosphoserine that has the following structure;

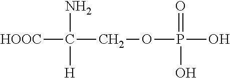 [SMILES: NC(COP(=O)(O)O)C(=O)O]

The compounds that are structurally similar to phosphoserine, which contain the reactive phosphonate or phosphate, and which have COOH functional groups, are capable of interacting with the Ca⁺⁺ within the TTCP to form a calcium based matrix and are referred to as compounds structurally similar to phosphoserine in this description. The combination of these functional groups plus the geometry properties of the matrix such as the chain length between the phosphorous and the COOH are unique aspects to the molecules which affect the level of adhesive bonding strength to substrate surfaces such as bone and metal.

The preferred compound that is structurally similar to phosphoserine is any form of phosphoserine, including the phospho-D-serine, phospho-L-serine or the phospho-DL-serine, and/or other similarly constructed compounds. The exact stereochemistry of the phosphoserine does not seem to have any impact on the properties of the compositions disclosed herein.

It has been found that when the quantity of the small amino acid phosphate species are included in the mixture and are increased beyond about 10% w/w based on the total composition of the dry ingredients, namely the metal compound, the small amino acid phosphate species and the bioactive glass material, more generally in the range of about 10% to about 90%, more typically in the range of about 15% to about 50%, or preferably from about 20% to about 40%, the tack and adhesion properties of the resulting compositions were significant. At such levels, the influence of the small amino acid phosphate species extends beyond internal interaction with the cement, but also extends to significant binding with the hydroxyapatite architecture and proteins of bone. At below about 10% by weight of the small amino acid phosphate species, the compositions do not have a tacky state and these compositions do not have adhesive properties.

Factors that may affect the length of the tacky state, the length of the putty states and the ultimate cure time, as well as strength properties of the compositions include: the percentage (w/w) multivalent metal compound and the small amino acid phosphate species based solely on the weight of the multivalent metal compound and the small amino acid phosphate species in the composition, the selection of the small amino acid phosphate species, the particle size of the multivalent metal compound, and the nature and quantity of any additives and/or fillers which may be combined to the composition to enhance the material properties.

The mean particle size of the multivalent metal compound should be greater than about 15 μm, preferably greater than about 25 μm. In one embodiment, the particle size may range from about 200 μm to about 400 μm. Further, the particle size distribution may be bimodal and include particles ranging in size from about 15 μm to about 25 μm and particles ranging in size from about 200 μm to about 400 μm. The bimodal particle size distribution may include about 20% to about 80% of one range and about 80% to about 20% of another, with the preferred distribution being about 50% of each size range. As the mean particle size of the multivalent metal compound is reduced, the multivalent metal compound tends to dissolve too fast and these compositions may not be practical for all uses as disclosed herein. On the other hand if the multivalent metal compound has a mean particle size of greater than about 1000 μm, the intra-operative performance of the compositions may not have the desired initial strength and be too slow to set. If a longer working time is desired, then multivalent metal compound with a larger mean particle size can be used; however, if a shorter working time is desired, then multivalent metal compound with a smaller mean particle sizes can be used. In certain use environments, compositions that have a multi-modal mean particle size distribution with, for example, one mode less then about 50 μm and the other mode above about 50 μm can provide unique properties such as a fast initial cure rate from the smaller mean particle size mode combined with higher intrinsic compression strength of the material from the larger mean particle size mode.

The aqueous based mixing media useful for combining the multivalent metal compound and the small amino acid phosphate species powders can include water, buffers such as sodium phosphate, saline, and blood based products such as whole blood, plasma, platelet rich plasma, serum, and/or bone marrow aspirate. The blood based products are used with the goal of achieving enhanced rate of bone healing and remodeling. It is also possible to use the compositions without premixing with an aqueous medium if the composition is to be used in a sufficiently wet environment that the aqueous medium can be absorbed from the in situ site. In this situation, the composition can be dusted on and/or other wise applied to the desired site and then mixed with the liquids that are already present at the site.

The amount of aqueous medium is not particularly important other than the amount should be chosen to provide the consistency of the desired product for use as a bone restoration composition or other use. The amount of aqueous medium present may range from about 10% to about 40% by weight. For example, the amount of aqueous medium present in a putty material may range between about 10% to about 20% by weight, whereas the aqueous medium present in a veneer, paintable material may range between 20% to about 40% by weight.

The bioactive glass used in the present disclosure may be any alkali-containing ceramic (glass, glass-ceramic, or crystalline) material that reacts as it comes in contact with physiological fluids including, but not limited to, blood and serum, which leads to bone formation. In preferred embodiments, bioactive glasses, when placed in physiologic fluids, form an apatite layer on their surface. The bioactive glass material of the present disclosure includes other suitable amorphous solid materials so long as the material has constituents that will interact with the calcium phosphate material as described herein.

The bioactive glass can be added to the formulation in the form of a coating, mono-block, fiber, flake, foam, granules, powders, and mixtures thereof. The bioactive glass material can be included directly within the cement formulation or can be an adjunct that is applied in situ as the cement is applied to the bone. The only important aspect is that sufficient bioactive glass material is available to ionically bond with the calcium phosphate and the adjunct material as described below.

In the present disclosure, a sodium phosphate compound may be used to speed the setting time of the bone cement. Examples of sodium phosphates which can be used, without limitation, are disodium hydrogen phosphate anhydrous, sodium dihydrogen phosphate monohydrate, sodium phosphate monobasic monohydrate, sodium phosphate monobasic dehydrate, sodium phosphate dibasic dehydrate, trisodium phosphate dodecahydrate, dibasic sodium phosphate heptahydrate, pentasodidium tripolyphosphate, sodium metaphosphate, and/or a mixture thereof. Examples of sodium phosphate compounds suitable for use in the present disclosure are described in U.S. Pat. No. 7,459,018, which is incorporated by reference herein in its entirety. In the preferred embodiment, the sodium phosphate compound is two sodium phosphate compounds, more preferably sodium phosphate dibasic anhydrous and sodium phosphate monobasic hydrate.

The particle size of the at least one sodium phosphate compound is between 1 μm to about 1000 μm. This means that at least about 25%, preferably about 50% and more preferably about 75%, of the sodium phosphate compound(s), by weight, falls within these ranges based on sieving. The sodium phosphate compound may be present in an amount of between 0.5% and about 5%, more preferably between about 0.5% and about 2.5%, based on the total weight of the total formulation.

In another preferred embodiment wherein the sodium phosphate compound is supplied as a liquid component, the sodium phosphate compound may be present in an amount of between 1% and about 10%, based on the total weight of the liquid component.

The bioactive glass material that is included within the composition should be sufficient to show a difference in intrinsic material strength (e.g. the bending moment energy to failure). That amount should be of sufficient weight within the formulation; amounts greater than about 0.1% w/w based on the total weight of the dry components of the composition would be sufficient. The bioactive glass material included within the composition can be up to about 75% w/w based on the total weight of the dry components of the composition. However, at such levels the adhesive properties decrease; therefore, a balance between intrinsic strength and material adhesive properties is required. More specifically, the intrinsic material strength of composition continuously increases as the amount of bioactive glass increases until the bioactive glass material included in the composition reaches 50% w/w based on total weight of the dry components of the composition. Once the amount of bioactive glass material exceeds 50% w/w based on total weight of the dry components, the composition becomes granular and the adhesive properties decrease. For bioactive glass materials in contact with the composition it is important that the surface area of the bioactive glass is readily available at the molecular level or is in direct contact with the composition.

It has been confirmed as noted above that the bioactive glass materials form a macromolecular network having a structure similar to what is shown in FIG. 1. In the illustrated network, the calcium ions from the TTCP form a non-covalent bond, such as an ionic bond, with the phosphoryl oxygen of the TTCP and the phosphoserine and also form ion-dipole bonds with the oxygen atoms and the calcium and silicon ions in both the bioactive glass material and pendant to the bioactive glass material. The bioactive glass molecules shown in FIG. 1 could represent either the molecules from the surface of a bioactive glass such as from a fiber added intrinsically to the composition or a molecule from the surface of an extrinsic bioactive glass source such as a bioresorbable bioactive glass plate.

The macromolecular networks can be formed at both an acidic and a basic pH. However, only when the pH is raised to a level above a pH of about 5 and preferably above a pH of about 6 so the macromolecular networks form permanent bonds. Also the pH should typically be below a pH of about 9 to be tolerated by the body. If the materials are to be used for other purposes, there is no upper limit other than a pH that will degrade the glass based material.

Cortical bone to bone shear strength of adhesive system containing glass beads showed higher strength than the adhesive system free of glass beads. The mechanical strength data and the FTIR data support the theory of the involvement of functional ions of glass fibers/beads in the formation of macromolecular network with the reactive phosphoserine and TTCP complex as shown in FIG. 1.

Additives may be included in the compositions disclosed herein to further enhance the material properties. These properties include the handling, porosity, intrinsic material strength, & bone healing rate (osteogenic). Depending on the multivalent metal compound chosen, suitable additives might include: alpha or beta tri-calcium phosphate (a-TCP or β-TCP), calcium sulfate, calcium silicate, calcium carbonate, sodium bicarbonate, sodium chloride, potassium chloride glycerol phosphate disodium, amino acids such as serine, excess amounts of phosphoserine, polyols (such as glycerol, mannitol, sorbitol, trehalose, lactose, & sucrose), silk, keratin (primarily found in human hair), autologous bone powder or chips, demineralized bone powder or chips, collagen, BMP7, stem cells, parathyroid hormone (PTH), bisphosphonates, and mixtures thereof. In addition, other additives and/or fillers could be incorporated which offer surgical visual aids & anti-infective properties. In a certain embodiments, no polymerizable material is included in the composition.

The α-TCP and β-TCP additive component typically is also in granular form. The granules presently contemplated have an overall diameter size in the range of about 0.1 to about 2 mm, or preferably between about 0.5 to about 1 mm. Larger and smaller granules can be used depending on the other components of the composition and the desired end properties. In the present compositions, the particle size of the granules has an impact on the mechanical strengths of the resultant compositions. The total porosity of these granules is in the range of about 40-80%, more preferably about 65-75%, and the average pore diameter size of the granules in these compositions is in the range of about 20-500 μm, preferably about 50-125 μm. The granules do not dissolve within the present embodiments during the curing phase, but interact as a solid particle with the other components of the compositions. In the present compositions, the porosity and pore size listed here has an impact on the resorption characteristics of the resultant compositions and to allow for bony in growth and healing as described by Dalai et al, in U.S. Pat. No. 6,949,251.

The additives that affect the porosity include cement curing pore forming agents such as calcium carbonate or sodium bicarbonate, granules with pre-formed pores made from alpha or beta tri-calcium phosphate (α-TCP or β-TCP), bioactive glasses that can open channels or pores as they degrade relatively quick in vivo. The rate at which the bioactive glasses degrade can be dependent on the physical state of the crystalline structure when processing the glass. Amorphous and partially amorphous glasses may resorb faster than crystalline materials of the same chemical composition, Small molecules may also be used which leach away relatively quickly from the cement as it cures; for example, these materials may include sodium chloride, potassium chloride, glycerol phosphate disodium, polyols (such as glycerol, mannitol, sorbitol, trehalose, lactose, & sucrose), amino acids such as serine, and/or excess amounts of phosphoserine. Other materials that form pores may dissolve or resorb over time in vivo and release from the cement opening pores; these materials include calcium sulfate, α-TCP or β-TCP powder or granules. Granules can be used to alter the in vivo resorption profile, such as α-TCP or β-TCP granules, or hybrid granules made from calcium sulfate and α-TCP or β-TCP in which the calcium sulfate portion resorbs more quickly.

The additives that affect the bone healing rate driven by new bone ingrowth can be influenced by the level of porosity of the cured cement. This rate can be manipulated by the number of pores and site of the pores created within the cured cement. Achieving such porosity up to about 60% v/v was demonstrated by controlling the ratio of composition ingredients. The porosity that develops during the curing process can be controlled by the amount of pore forming agent added (such as calcium carbonate), the level of compound structurally similar to phosphoserine added, the level of aqueous solution used, and/or the level of other agents added to the composition. Increasing the porosity reduces the material intrinsic strength; however, a balance of porosity vs. strength is critical for achieving the clinical application. Additives that increase the intrinsic material strength can be incorporated to offset the loss of strength by creating porosity.

These glass based materials can be supplied as fibers, powders, or any other suitable forms into the composition to increase the material intrinsic strength. An important aspect for chemical ion-dipole adhesion of these fibers is the size and/or surface area. The size or surface area can be defined by the aspect ratio (length:diameter). The preferred aspect ratio is from 2:1 to 50:1; more preferable from 10:1 to 35:1. The overall length of the fiber can be up to 5 mm; however, since the material could be used as bone to bone adhesive, the length of the fiber may be more appropriate at lengths up to 2 mm.

The additives that act as visual aids in the surgical procedure include colorants such as a pigment or dye to aid in determining coverage and depth of the applied cement or contrast agents such as barium salts in determining depth on a radiograph.

Other additives can be incorporated into the compositions that enhance the bone healing rate (osteogenic). These additives comprise a class of osteogenic growth factors including bone morphogenetic proteins (BMP's), such as BMP 7, stem cells, parathyroid hormone (PTH) and/or anti-osteoporotic agents such as bisphosphonates can be contemplated for incorporation into the composition.

Other additives that can be incorporated into the composition are infection preventatives such as broad spectrum antibiotics and anti-infection additives.

The compositions as described herein have many unique properties not found in prior calcium phosphate compositions. One particularly important property is that the compositions have a tacky state immediately subsequent to mixing with an aqueous medium. This tack property is retained for a number of minutes, sometimes up to about 12 minutes depending on the application requirement, typically up to about 4 minutes, and preferably up to about 2 minutes, after mixing with the aqueous medium. The time of the tacky state is dependent on a number of factors including relative ratio of the components, the particle sizes of the component materials, the presence of additives and the like. During this time the compositions will adhere bone to bone and bone to other materials, often without the need for external clamping or other application of pressure. The tacky state is not so aggressive that the composition will permanently affix the materials together at this point in time. Rather the tacky state can allow the materials to be moved relative to each other and also to be re-opposed without appreciable loss of ultimate cured strength. This is important in a medical setting so that the user can make sure the bone and the other material to be adhered to the bone are in the proper position relative to each other.

The tacky state is followed by a putty state. In the putty state, the tacky property has substantially disappeared and the compositions can be shaped or sculpted. In addition, during the putty state, the composition can be formed into shapes or used to fill voids in bone in a manner similar to putty. This putty state is retained for a number of minutes, sometimes up to 15 minutes depending on the application requirement, typically up to about 8 minutes, and preferably up to about 5 minutes, after mixing with the aqueous medium. Like the tacky states, the putty state is dependant on a number of factors including the relative ratio of the components, the presence of additives, the particle size of the components and the like.

Because the items to be affixed can be repositioned during the tacky state or the compositions can be shaped during the putty state, this combined time of the tacky state and the putty state is some times referred to as the working time. Typical compositions have a working time of up to about 8 minutes from initial mixing and often the working time is up to about 5 minutes after which time the compositions have sufficiently begun hardening that further manipulation will result in degradation of ultimate strength of the bond.

After the putty state, the compositions harden like a cement to form a substantially permanent bond between the materials. The bond is made stable initially due to the adhesive properties of the composition. The bond is maintained over time, in vivo, due to bone ingrowth into the composition and materials concurrent with any resorption of the compositions and materials. In the cement state, the composition hardens and the materials that have been affixed to each other cannot be separated without the application of significant force. The compositions typically will begin to harden within about 12 minutes, and often within about 7 minutes, after mixing with the aqueous medium. The amount of time to reach the cement state is also dependant of the same factors listed above.

It should be understood that in certain embodiments of the present invention, for instance in applications in which the composition serves mainly as an adhesive, it is not desirable to work the product through a putty state. For these applications, the composition is optimized to include a tacky state with little or no putty state. In these embodiments, the working time is predominantly the tacky state (and shortened) and can range from up to about 2 minutes to up to about 12 minutes after mixing with an aqueous medium.

A further important property of the compositions is that these compositions have significant coherency and integrity within a wet environment. In the medical field, this would include a surgical site and a wound or similar situation where blood and other bodily fluids are present. The tacky state, the putty state and the cement state are not inhibited by environment. Specifically, all can transpire in either a wet environment or in a dry environment. In order to get the desirable properties, the user need not ensure that the application site is clean and dry. In a wet environment, the compositions tend to remain together and the presence of the liquid does not significantly affect the integrity of the composition or the ultimate strength properties. In certain embodiments in fact, it is preferred that the local aqueous medium (such as blood, bone marrow) be incorporated into the composition.

While not wishing to be bound by theory, compositions of the present disclosure are believed to function as follows: the TTCP, which is basic in nature, reacts with the small amino acid phosphate species, which is acidic in nature, upon mixing with the aqueous medium and forms a hardened, layered structure upon curing. This reaction is exothermic; the degree of exothermic activity depends on a number of factors including the volume of the composition. The low pH nature of the compounds that are structurally similar to phosphoserine enable the hydroxyl of phosphate or phosphonate and COOH functional group to bond through ionic interaction with the calcium ions from within the TTCP. This resulting reactive intermediate continues a cascade of ionic interactions with calcium and phosphate ions within the TTCP or HA on the bone surface or any other metal ions of the metal implants. This series of interactions provides transient material having the tacky properties while curing and the adhesion strength that increases upon cure.

The exothermic properties of the composition when curing are prevalent when mixing as a large volume bone void filler (usually greater then 10 cc) and this may serve as an effective means to kill the residual tumor cells locally that remain after surgical bone tumor removal.

The exothermic properties of the composition may lead to necrosis of local tissue and this also reduces the adhesive working time. The amount of heat released by the exothermic reaction is mainly influenced by the volume of the composition, the size of the particles and the ratio of compound that is structurally similar to phosphoserine to TTCP. With larger volumes of composition, more heat is released to the surrounding tissue. With volumes less than or equal to about 1 cc, the heat release is negligible with maximum temperature reached during the curing of the adhesive being below about 40° C. The higher volume compositions greater than about 1 cc, led to considerable heat release, even exceeding about 60° C. in compositions greater than about 5 cc. To manage this exothermic heat release to below about 45° C., the particle size distribution of the TTCP and the ratio of TTCP to compound that is structurally similar to phosphoserine can be chosen appropriately. The smaller TTCP particles dissolve and react faster due to a higher specific surface area; therefore, to reduce the exothermic heat release, the composition can be adjusted by choosing a TTCP particle size distribution which generally has a mean particle size greater than about 15 μm, preferably greater than about 25 μm. As noted above, one embodiment may include particle sizes ranging from about 200 μm to about 400 μm. Other embodiments may include a bimodal particle size distribution comprising particles ranging in size from about 15 μm to about 25 μm and particles ranging in size from about 200 μm to about 400 μm. In addition, the greater amount of TTCP to the compound that is structurally similar to phosphoserine used, the faster the reaction occurs due to the number of calcium ions available for bonding. Exothermic heat release can be limited by adding more compound that is structurally similar to phosphoserine to the composition. To further reduce the exothermic heat release, endothermic additives can be incorporated into the composition to slow the reaction rate; these include polyols (such as sorbitol or mannitol) and/or PEG. The factors discussed here can be chosen to design several compositions; all of which have exothermic profiles which limit or eliminate necrotic reactions to local tissues while tailoring the compositions with sufficient working time for the clinical application.

The compositions when mixed with aqueous medium typically have a creamy or a tacky paste consistency initially. Also, the mixing of the compositions with the aqueous medium does not require a high level of force or shear. Generally, simple hand mixing, such as with a spatula, is sufficient in most instances. It is envisioned that the present compositions may be applied via injection through a syringe or other suitable pressurized implement, applied with a spatula, and as otherwise desirable by a user. The creamy or tacky viscosity allows for application of the composition to the defect site for a defined period of time. The compositions allow the bone to be repositioned several times within about 4 minutes and preferably within about 2 minutes without losing tack properties. If the compositions need to be injected through a syringe or cannula, the viscosity of the composition during the working time can be important. For these situations, viscosities of the compositions herein should be preferably below about 150 centipoise.

Still further embodiments have a consistency similar to putty. These embodiments are useful for filling larger defects, have sculpting properties, or for mechanical interlocking into cancellous bone. These compositions hold their cohesive, tacky, and sculpting properties over a longer period of time even when subjected to a wet field. The compositions have working time for sculpting sometimes up to about 15 minutes depending on the application requirement, typically up to about 8 minutes, and preferably up to about 5 minutes, after mixing with the aqueous medium. Formulations with an increased amount of small amino acid phosphate species greater than about 25% w/w or increased TTCP mean particle size greater than about 250 microns tend to have longer working times and seem to be suitable for use in situations were the putty will fill defects in structures that are well supported by surrounding bone. In these situations the putty does not need to harden as fast provided it maintains its cohesive properties in the wet field. Another property of the compositions is that the compositions will adhere to themselves as well as to an external surface such as bone. This is useful in situations where a shape is formed during the putty state and this shape can then adhere to bone. Also, in some instances a user may apply a mass of the composition to a bone or other surface and then shape the composition into the final desired shape during the working time of the composition.

Compositions which have a putty consistency to be used as a void filler can be enhanced by incorporating macro porous granules or chips to allow for new bone ingrowth. These granules may come from synthetic sources such bioactive glass (partial, or wholly amorphous), α-TCP or β-TCP granules or it may be preferred to select the granules or chips from autologous bone sources or demineralized bone to enhance the bone healing rate.

Additional embodiments have a consistency that is thin, free flowing, and paintable. The increased amount of aqueous medium does not detract from the adhesive strength in the tacky state. These embodiments are useful for painting or coating on the surface of an implant prior to insertion into a bone structure, which significantly increases the pull out strength of the implant from the bone structure. It is believed that the application of this embodiment on an implant prior to insertion into a bone structure prevents micro motions of the implant shortly after the implant is put in position in the patient and minimizes the implant failure.

It is further envisioned that the cement compositions disclosed herein may be packaged into kits that may include a vial containing the TTCP with the small amino acid phosphate species and the bioactive glass material pre-filled together and packaged under vacuum, nitrogen, or dry air to preserve the shelf life. Further, if additives are used, they may be included within this vial or in a separate vial. The aqueous medium is provided in a separate vial. The kit may include mixing bowls, stirring sticks, spatulas, syringes, and/or any other desirable component for application.

Composition of the current disclosure are envisioned to provide ease of use in different medical applications based on ease of application, duration of use before cure, resistance to in vivo environments, extended maneuverability of bone fragments and/or implant devices prior to cure onset, good load bearing capabilities after cure, and good ultimate bond strength. For example, compositions may have an adequate working period after mixing sometimes up to about 15 minutes depending on the application requirement, typically up to about 8 minutes or less, and preferably up to about 5 minutes or less. Further, the relative force of pressure required to inject the composition from an appropriately sized syringe may remain constant or below a certain injection force threshold from the point of mixing and loading the syringe to the end of the working period. It is contemplated that bone fragments adhered together or implanted devices may exhibit moderate separation strengths within the working period. Such moderate separation strengths may be exhibited regardless of the relative compressive force used during apposition. It is further contemplated that cement compositions of the present disclosure may have sufficient material cohesion when applied in moist, wet, greasy and/or fatty saline environments, such as in vivo settings, thereby reducing the need for surface preparation and maintaining a dry environment. As well, good capacity for supporting passive movement and maintaining load and non-load bearing bone fragment alignment after surgery during initial rehabilitation period and active range of motion rehabilitation period are envisioned for cement compositions contemplated herein.

Typical compositions exhibit an adhesive strength upon curing, typically after greater than about 10 minutes from initial mixing, in the range of about 250 to about 2000 kPa on cancellous bone and from about 250 to about 10,000 kPa on cortical bone in at least one of compression, tension, shear, and/or bending. Compositions can be chosen to achieve the strength in these ranges; the level of strength required is dependent upon the clinical application. Also, it is important to note that the curing can be accomplished either in a wet environment, such as in bodily fluids, or in a dry environment, and the ultimate strength of the bond after cure does not seem to be significantly affected.

### Examples 1-42

In Table 1, the shear testing was done using an Instron Force test machine (Model #5564) setup as follows. The sample was supported and fastened to the machine at one end of the sample and the other end was left free and unsupported. Each sample had a bond surface that was 90 to the face of the bone samples. The force test probe was placed in plane against the top of the bond line of the sample and force was applied until failure. When the bond failed, the result was recorded. The TTCP that was used in Examples 1-12 was a commercially available material that included from about 17% to 32% of related impurities. These materials all contained about 68% to 83% TTCP.

In order to see the effect on the intrinsic material strength, bioactive glass material was added to the compositions to form a macromolecular network. Referring to Table 1, a cement formulation having the following formulation: 1.6 g TTCP, 1 g Phosphoserine, and various amounts of bioactive glass and water was mixed and applied between two separate 10 mm×10 mm cortical bone cubes. Before strength testing, the samples were allowed to cure for 10 minutes submersed in water at 32° C.

TABLE 1
Cortical Bone to Bone Shear Strength of
TTCP/Phosphoserine with Bioactive Glass By Weight

| | | | Combeite | Combeite | | |
|---|---|---|---|---|---|---|
| | | | Bioactive Glass | Bioactive | | |
| | TTCP | Phosphoserine | 32-90 microns | Glass 90-710 | Water | Shear Strength |
| Ex. | (g) | (g) | (g) | microns (g) | (μl) | (MPa) |
| 1 | 1.6 | 1 | — | — | 532 | 1.23 (n = 3) |
| 2 | 1.6 | 1 | 0.12 | — | 532 | 1.347 (n = 3) |
| 3 | 1.6 | 1 | 0.24 | — | 532 | 1.237 (n = 3) |
| 4 | 1.6 | 1 | 0.40 | — | 560 | 1.22 (n = 3) |
| 5 | 1.6 | 1 | 0.8 | — | 590 | 1.48 (n = 3) |
| 6 | 1.6 | 1 | 1.2 | — | 640 | 1.11 (n = 3) |
| 7 | 1.6 | 1 | 1.2 | — | 720 | 1.037 (n = 3) |
| 8 | 1.6 | 1 | 1.6 | — | 1000 | 0.80 (n = 3) |
| 9 | 1.6 | 1 | — | 0.12 | 532 | 0.753 (n = 3) |
| 10 | 1.6 | 1 | — | 0.40 | 560 | 1.347 (n = 3) |
| 11 | 1.6 | 1 | — | 0.80 | 590 | 1.91 (n = 3) |
| 12 | 1.6 | 1 | — | 1.6 | 650 | 2.13 (n = 3) |

A small amount of each of the above formulations was applied both to a cortical bone that has been clamped in place and to the appropriate cortical bone cube and tested using a shear testing device with a force vector (F) applied directly to the cement bond. With respect to the formulations comprising combeite glass sized from 32-90 microns, the formulation of Example 5 had substantially higher shear strength than the Comparative Examples 1-4 and 6-8. With respect to the formulations comprising combeite glass sized from 90-710 microns, the formulation of Example 12 had substantially higher shear strength than the Comparative Examples 9-11.

### Examples 13-16

In certain embodiments of the present invention, certain small molecule amino acid phosphate compounds such as phosphoserine, having a phosphate group (PO₄), a carboxyl group (COOH), and an amine group (NH₂), form ionic interactions with available metal ions. As shown in Table 2, these metal ions may be provided via the use of various calcium salts and calcium-based materials including CaO, CaCl₂ or Ca(OH)₂, Table 2 shows certain non-limiting formulations that may be used in the present invention,

TABLE 2
Cortical Bone to Bone Shear Strength of Calcium-Based
Materials/Phosphoserine with Bioactive Glass By Weight

| | | | Combeite | | |
|---|---|---|---|---|---|
| | | Phospho- | Bioactive | | |
| | Calcium | serine | Glass 90-710 | Water | |
| Ex. | Salt | (g) | microns (g) | (ml) | Material Property |
| 13 | 0 | 1 | 1.6 | 0.528 | Forms sticky mass, |
| | | | | | but didn't cure |
| 14 | Ca(OH)₂, | 1 | 1.6 | 0.528 | Forms sticky mass, |
| | 140 mg | | | | and cured by itself |
| 15 | Ca(OH)₂, | 1 | 1.6 | 0.6 | Forms sticky mass, |
| | 240 mg | | | | putty and cured by |
| | | | | | itself |
| 16 | CaCl₂, | 1 | 1.6 | 0.6 | Forms sticky mass, |
| | 240 mg | | | | putty and cured by |
| | | | | | itself |

In these formulations, the phosphoserine molecule itself initiates reactions with the ionic composition of glass fibers and forms a sticky mass. With an insufficient amount of the calcium-based material, the formulation does not fully cure in part due to calcium deficiency and low pH. The use of calcium salts such as CaO, CaCl₂ and Ca(OH)₂ facilitates curing of the sticky mass.

### Example 17

In a further example utilizing a calcium-based material, 0.040 g CaO was mixed with 1 g Phosphoserine, 1.6 g Combeite (90-710 microns), and 0.528 mL water. The formulation formed a sticky putty that cured into a solid mass with potential load bearing applications. The strength testing of Example 17 was performed in accordance with Examples 1-12 above, Example 17 had a shear strength of 0.21 MPa.

The formulations of Examples 13-47 are most suitable for use as bone void fillers, bone graft materials and in other orthopedic applications with minimum load bearing requirements.

### INDUSTRIAL APPLICATION

Cement compositions disclosed herein provide adhesive and cohesive strength through mechanical and chemical interlocking with bone substrates. The cement formulations have a tack and/or sticky quality that allows temporary adherence early in the curing process and a delay in significant curing early on. The cement formulations may mimic natural bone architecture and provide superior mechanical strength over longer periods of time relative to convention cement formulations.

The disclosure has been presented in an illustrative manner in order to enable a person of ordinary skill in the art to make and use the disclosure, and the terminology used is intended to be in the nature of description rather than of limitation. It is understood that the disclosure may be practiced in ways other than as specifically disclosed, and that all modifications, equivalents, and variations of the present disclosure, which are possible in light of the above teachings and ascertainable to a person of ordinary skill in the art, are specifically included within the scope of the impending claims. All patents, patent publications, and other references cited herein are incorporated herein by reference.

What we claim is:

1. A bone restorative composition comprising a reactive mixture of a small amino acid phosphate species, a multivalent metal compound, and a bioactive glass material that contains ionic functional groups.

2. The bone restorative composition of claim 1, wherein the small amino acid phosphate species comprises a compound of the formula; 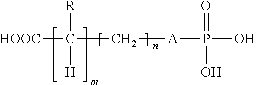
where A is O, CH₂, or S; R is H, NH₂, NHCO(CH₂)ₜCH₃ where t is 0 to 2, NH(CH₂)ₓCH₃ where x is 0 to 3, NR1R2 where R1 is (CH₂)_{y}CH₃ and R2 is (CH₂)_{y}CH₃ where y is 0 to 2, (CH₂)_{z}CH₃ where z is 0 to 3, where m is 0 to 1, and where n is 0 to 3;

3. The bone restorative composition of claim 1, wherein the small amino acid phosphate species is present in an amount from about 10% to about 90% by weight, preferably about 15% to about 50% by weight, and more preferably about 20% to about 40% by weight, based on the combined weight of the compound, the multivalent metal compound, and the bioactive glass material.

4. The bone restorative composition of claim 1, wherein the small amino acid phosphate species is phosphoserine.

5. The bone restorative composition of claim 1, wherein the multivalent metal compound is present in an amount from about 5% to about 90% by weight, preferably about 40% to about 80% by weight, and more preferably about 40% to about 65% by weight, based on the combined weight of the small amino acid phosphate species, the multivalent metal compound, and the bioactive glass material.

6. The bone restorative composition of claim 1, wherein the multivalent metal compound is selected from the group consisting of tetracalcium phosphate, calcium oxide, calcium chloride, or calcium hydroxide.

7. The bone restorative composition of claim 1, wherein multivalent metal compound has a mean particle size of greater than 15 μm, preferably greater than 25 μm.

8. The bone restorative composition of claim 1, wherein multivalent metal compound has a mean particle size between about 200 μm and about 400 μm.

9. The bone restorative composition of claim 1, wherein the multivalent metal compound has a bimodal particle size distribution that includes particles ranging in size from about 15 μm to about 25 μm and particles ranging in size from about 200 μm and 400 μm.

10. The bone restorative composition of claim 1, wherein the bioactive glass material is present in an amount from about 0.1% to about 75% by weight, preferably about 0.1% to about 50% by weight, based on the combined weight of the small amino acid phosphate species, the multivalent metal compound, and the bioactive glass material.

11. The bone restorative composition of claim 1, wherein the bioactive glass material is comprised of particles ranging in size from about 0.1 to about 710 microns.

12. The bone restorative composition of claim 11, wherein the bioactive glass material is comprised of particles ranging in size from about 32 to about 90 microns.

13. The bone restorative composition of claim 11, wherein the bioactive glass material is comprised of particles ranging in size from about 90 to about 710 microns.

14. The bone restorative composition of claim 11, wherein the bioactive glass material has a bimodal particle size distribution that includes particles ranging in size from about 32 to about 90 microns and particles ranging in size from 90 to about 710 microns.

15. The bone restorative composition of claim 1, wherein the bioactive glass material is supplied as fibers or powders.

16. The bone restorative composition of claim 1, further comprising an aqueous medium.

17. The bone restorative composition of claim 16, wherein the composition has a pH at a level between about 5 and about 9, preferably between about 6 and about 9.

18. The bone restorative composition of claim 16, wherein the bone restorative composition has a shear strength of at least 0.20 MPa upon curing.

19. The composition of claim 16, wherein the aqueous medium is present in an amount from about 10% to about 40% by weight of the total composition.

20. The bone restorative composition of claim 16, wherein the aqueous medium is water.

21. The bone restorative composition of claim 16, wherein the aqueous medium is a blood-based product.

22. The composition of claim 16, wherein the composition has a tacky state for up to about 12 minutes, preferably for up to about 4 minutes, and most preferably for up to about 2 minutes, after mixing with the aqueous medium.

23. The composition of claim 22, wherein the composition during the tacky state has a separation strength in the range of about 10 kPa to about 250 kPa, and preferably in the range of about 50 kPa to about 150 kPa, after mixing with the aqueous medium.

24. The composition of claim 16, wherein the composition has a putty state for up to about 15 minutes, preferably up to about 8 minutes, and most preferably up to about 5 minutes, after mixing with the aqueous medium.

25. The composition of claim 16, wherein the composition has a working time for up to about 15 minutes, preferably up to about 8 minutes, and more preferably up to about 5 minutes, after mixing with the aqueous medium.

26. The bone restorative composition of claim 1, further comprising an additive.

27. The bone restorative composition of claim 26, wherein the additive is selected from the group comprising alpha or beta tri calcium phosphate (α-TCP or β-TCP), calcium sulfate, calcium silicate, calcium carbonate, sodium bicarbonate, sodium chloride, potassium chloride glycerol phosphate disodium, amino acids such as serine, excess amounts of phosphoserine, polyols (such as glycerol, mannitol, sorbitol, trehalose, lactose, & sucrose), silk, keratin (primarily found in human hair), autologous bone powder or chips, demineralized bone powder or chips, collagen, BMP7, stem cells, parathyroid hormone (PTH), bisphosphonates, and mixtures thereof.

28. The composition of claim 26, wherein the additive is a reaction rate modifier.

29. The composition of claim 26, wherein the additive is a pore former.

30. The composition of claim 26, wherein the additive enhances resorption.

31. The composition of claim 26, wherein the additive is a strength modifier.

32. The composition of claim 26, wherein the additive promotes bone healing.

33. The composition of claim 26, wherein the additive is a contrast agent.

34. A kit for restoring bone comprising;
A) a first container containing a composition comprising:
(i) a small amino acid phosphate species comprising a compound of the formula; 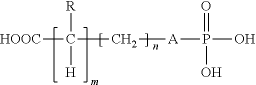
where A is O, CH₂, or S; R is H, NH₂, NHCO(CH₂)ₜCH₃ where t is 0 to 2, NH(CH₂)ₓCH₃ where x is 0 to 3, NR1R2 where R1 is (CH₂)_{y}CH₃ and R2 is (CH₂)_{y}CH₃ where y is 0 to 2, (CH₂)_{z}CH₃ where z is 0 to 3, where m is 0 to 1, and where n is 0 to 3;
(ii) a multivalent metal compound; and
(iii) a bioactive glass material that contains ionic functional groups; and
B) a second container containing an aqueous medium.

35. The kit of claim 34, further including a delivery device that mixes the contents of the first and second containers.

36. The kit of claim 34, wherein the small amino acid phosphate species is present in an amount from about 10% to about 90% by weight, preferably about 15% to about 50% by weight, and more preferably about 20% to about 40% by weight, based on the combined weight of the of the compound, the multivalent metal compound, and the bioactive glass material.

37. The kit of claim 34, wherein the small amino acid phosphate species is phosphoserine.

38. The kit of claim 34, wherein the multivalent metal compound has a mean particle size of greater than about 15 μm, preferably greater than about 25 μm.

39. The kit of claim 34, wherein multivalent metal compound has a mean particle size between about 200 μm and about 400 μm.

40. The kit of claim 34, wherein the multivalent metal compound has a bimodal particle size distribution that includes particles ranging in size from about 15 μm to about 25 μm and particles ranging in size from about 200 μm and 400 μm.

41. The kit of claim 34, wherein the bioactive glass material is present in an amount from about 0.1% to about 75% by weight, preferably about 0.1% to about 50% by weight, based on the combined weight of the small amino acid phosphate species, the multivalent metal compound, and the bioactive glass material.

42. The kit of claim 34, wherein the bioactive glass material is comprised of particles ranging in size from about 0.1 to about 710 microns.

43. The kit of claim 42, wherein the bioactive glass material is comprised of particles ranging in size from about 32 to about 90 microns.

44. The kit of claim 42, wherein the bioactive glass material is comprised of particles ranging in size from about 90 to about 710 microns.

45. The kit of claim 42, wherein the bioactive glass material has a bimodal particle size distribution that includes particles ranging in size from about 32 to about 90 microns and particles ranging in size from 90 to about 710 microns.

46. The kit of claim 34, wherein the aqueous medium is a blood based product.

47. The kit of claim 34, wherein the aqueous medium is water.

48. The kit of claim 34, wherein the composition further includes an additive.

49. The kit of claim 48, wherein the additive is selected from the group consisting of alpha tri-calcium phosphate, beta tri-calcium phosphate, calcium sulfate, calcium silicate, calcium carbonate, sodium bicarbonate, sodium chloride, potassium chloride, glycerol phosphate disodium, amino acids, polyols, trehalose, lactose, sucrose, silk, keratin, autologous bone powder or chips, demineralized bone powder, demineralized bone chips, collagen, biodegradable polymers, bone morphogenetic protein 7, stem cells, parathyroid hormone, bisphosphonates, and mixtures thereof.

50. The kit of claim 48, wherein the as additive is a reaction rate modifier.

51. The kit of claim 48, wherein the as additive is a pore former.

52. The kit of claim 48, wherein the additive enhances resorption.

53. The kit of claim 48, wherein the additive is a strength modifier.

54. The kit of claim 48, wherein the additive promotes bone healing.

55. The kit of claim 48, wherein the additive is a contrast agent.

56. A method of adhering a substance to a bioactive glass material comprising the steps of:
placing a composition between the substance and the bioactive glass material wherein the composition comprises:
(i) a small amino acid phosphate species comprising a compound of the formula: 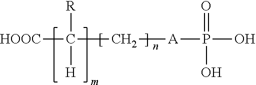
where A is O, CH₂, or S; R is H, NH₂, NHCO(CH₂)ₜCH₃ where t is 0 to 2, NH(CH₂)ₓCH₃ where x is 0 to 3, NR1R2 where R1 is (CH₂)_{y}CH₃ and R2 is (CH₂)_{y}CH₃ where y is 0 to 2, (CH₂)_{z}CH₃ where z is 0 to 3, where m is 0 to 1, and where n is 0 to 3,
(ii) a multivalent metal compound; and
(iii) an aqueous medium; and
allowing the composition to cure to form an macromolecular network at the interface between the composition and the bioactive glass material,
wherein the bioactive glass material contains ionic functional groups as the bonding sites of the bioactive glass material, and wherein the composition adheres to the substance.

57. The method of claim 56, wherein the substance is an implant.

58. The method of claim 56, wherein the substance is a bone.

59. The method of claim 58, wherein the mixture is applied to a void in the bone to fill the void.

60. The method of claim 56, wherein the applying step further comprises coating or painting the bone with the composition.

61. The method of claim 56, wherein the aqueous medium is a blood based product.

62. The method of claim 61, wherein the composition combines in situ with the blood based product.

63. The method of claim 56, wherein the aqueous medium is water.

64. A method of repairing a hard surface comprising the steps of:
mixing a composition comprising an effective amount of multivalent metal compound, a bioactive glass material, and a small amino acid phosphate species, wherein the small amino acid phosphate species comprising a compound of the formula: 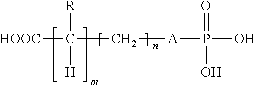
where A is O, CH₂, or S; R is H, NH₂, NHCO(CH₂)ₜCH₃ where t is 0 to 2, NH(CH₂)ₓCH₃ where x is 0 to 3, NR1R2 where R1 is (CH₂)_{y}CH₃ and R2 is (CH₂)_{y}CH₃ where y is 0 to 2, (CH₂) CH₂ where z is 0 to 3, where m is 0 to 1, and where n is 0 to 3,
with an aqueous medium to create a mixture;
applying the mixture to the hard surface to be repaired; and
allowing the mixture to cure.

65. The method of claim 64, wherein the hard surface is an implant.

66. The method of claim 64, wherein the hard surface is a bone.

67. The method of claim 66, wherein the mixture is applied to a void in the bone to fill the void.

68. The method of claim 64, wherein the applying step further comprises coating or painting the hard surface with the composition.

69. The method of claim 64, wherein the aqueous medium is a blood based product.

70. The method of claim 69, wherein the composition combines in situ with the blood based product.

71. The method of claim 64, wherein the aqueous medium is water.
